(19) United States
(12) Patent Application Publication
(10) Pub. No.: US 2006/0149084 A1
(43) Pub. Date: Jul. 6, 2006

(54) Method for extracting 2-ketone-l-gulonic acid from a polar, preferably aqueous solvent

(76) Inventors: Thomas Domschke, SPEYER (DE); Martin Merger, Ludwigshafen (DE); Petra Deckert, Wiesloch (DE); Friedrich Sauer, Obersulzen (DE)

Correspondence Address: CONNOLLY BOVE LODGE & HUTZ, LLP, P O BOX 2207, WILMINGTON, DE, 19899, US

(21) Appl. No.: 10/539,960

(22) PCT Filed: Dec. 13, 2003
(86) PCT No.: PCT/EP03/14192

§ 371 Date: Jun. 17, 2005

(30) Foreign Application Priority Data
Dec. 19, 2002 (DE) 102 60 085.6
Apr. 8, 2003 (DE) 103 16 268.2

(51) Int. Cl. C07D 307/62; C07C 51/245

(52) U.S. Cl. 549/315; 562/527

(57) ABSTRACT

The present invention relates to a process for extracting 2-keto-L-gulonic acid (KGA) from a polar, preferably aqueous, solvent, preferably from a solvent which comprises a mixture of ascorbic acid and 2-keto-L-gulonic acid, by means of liquid/liquid extraction using an extractant which comprises a tertiary amine and a polar organic diluent. The process of the invention preferably also comprises steps for back-extraction of the KGA and recycling of the extractant. The present invention likewise relates to a process for preparing ascorbic acid from KGA and isolating the prepared ascorbic acid.

L-Ascorbic acid (vitamin C, ascorbic acid, L-xylo-ascorbic acid, L-threo-hex-2-enonic acid γ-lactone) is normally prepared from 2-keto-L-gulonic acid (KGA), monoacetone-2-keto-L-gulonic acid or diacetone-ketogulonic acid. In newer processes, KGA is obtained in a one-stage or a multistage fermentation process, for example by two-stage fermentation of sorbitol via sorbose using microorganisms suitable for this purpose, some of which have been specifically modified.

KGA, or the diacetone-2-keto-L-gulonic acid resulting from the “Reichstein process”, is lactonized directly or via intermediates such as, for example, esters, especially methyl or butyl esters. Acids, usually mineral acids, especially concentrated hydrochloric acid (acid lactonization) or bases such as, for example, sodium hydroxide solution, NaHCO₃, Na₂CO₃, alcoholates etc. (alkaline lactonization) are employed as catalyst. The autocatalytic conversion of KGA to ascorbic acid has also been described. The product resulting from the lactonization reaction is crude ascorbic acid with a variable content of KGA, from which the ascorbic acid must then be purified.

Various separation processes have been described in the literature. Economic fractionation of a mixture of ascorbic acid and KGA is in principle rather difficult. Ascorbic acid and KGA differ in their chemical structure only by the lactone structure of ascorbic acid formed during the lactonization. Accordingly, they resemble one another in their chemical reaction properties and have similar physical properties. Thus, both acids show a comparable pH- and temperature-dependent tendency to decompose and form colored subsidiary components under the usual industrial preparation and purification conditions. The solubility of KGA and ascorbic acid is determined by the four hydrophilic hydroxyl groups and the acid group. Both have a similar solubility product in polar solvents: they are readily soluble in polar solvents, especially water, but only slightly soluble in nonpolar organic media.

This is evident in particular in the processes for separating ascorbic acid from the precursor KGA or its derivatives described in the prior art for the preparation of ascorbic acid.

According to JP 85019285, ascorbic acid and KGA can be separated from one another from aqueous solution by crystallization of the KGA as Na KGA. It is then necessary to liberate KGA from Na KGA in a subsequent step.

Crystalline ascorbic acid is also provided by the process of JP 31856. The publication describes acid-catalyzed lactonization of diacetone-2-keto-L-gulonic acid hydrate in a mixture of toluene, an alcohol and acetone as solvents.

In DE 641639, halohydrocarbons are added as precipitation aids in order to achieve adequate yields and ascorbic acid purities. This results in unwanted byproducts such as alkyl halides, which require elaborate disposal.

Alkali-catalyzed processes initially result in the sodium salt of ascorbic acid, which must be converted in a further process step into free ACA and is associated with equimolar production of NaCl or Na₂SO₄. A further crystallization step is usually necessary thereafter.

A process which provides free ascorbic acid without production of salts is described in U.S. Pat. No. 5,041,563. This proposes base-catalyzed lactonization of a KGA ester using a long-chain amine in a dipolar solvent to give an ammonium ascorbate. The liberation of ascorbic acid is then brought about by extraction of the amine with a nonpolar solvent. At the same time, colored byproducts are also extracted.

Catalyst-free methods for synthesizing ascorbic acid from KGA esters have been known since about 1940.

DE 861 841 describes a direct lactonization with partial conversion and ascorbic acid removal by selective crystallization and precursor recycling. Unreacted precursor is removed by crystallizing the ascorbic acid. The precursor must be present only in low concentration in the mother liquor after the crystallization because, otherwise, the product is contaminated. It is therefore necessary to operate with high conversions.

U.S. Pat. No. 1,904,619 describes a process for continuous KGA (derivative) lactonization with partial conversion in aqueous solution. The product is isolated by crystallization and recrystallization from methanol. All the mother liquors must be combined, concentrated and converted back into an aqueous solution.

WO 98/08584 deschribes a process for liquid/liquid extraction of acids from aqueous solutions with supercritical CO₂. However, it is used only for isolating pure KGA or ascorbic acid from a fermentation broth. Selective separation of KGA and ACA from an aqueous solution is not described.

According to FR 1050832 and FR 1099614, impurities are extracted from aqueous solution by liquid/liquid extraction and thus separation of ascorbic acid from sugars is achieved, and purification of crude ascorbic acid is made possible.

No economic processes for separating ascorbic acid and KGA have yet been made available in the state of the art, so that in the processes for preparing ascorbic acid ordinarily the lactonization must be carried out with complete conversion of the KGA or the particular precursor, in order to avoid contamination of ascorbic acid with KGA.

In many processes there is derivatization of the precursor or product. Thus, there is in particular the formation of methyl or butyl esters of KGA, which are soluble in alcohol in contrast to ascorbic acid. The described separation processes are very complicated and of low efficiency. They are moreover ecologically unsatisfactory because of the high consumption of energy and the use of organic solvents, most of which are toxic.

The preparation of ascorbic acid is, however, distinguished by special requirements for the purity and yield in all stages of the process: firstly to make it possible to use the final product in human nutrition applications, and secondly to minimize the costs of preparation.

It is an object of the present invention to provide an advantageous process to allow 2-keto-L-gulonic acid to be separated selectively and economically from a mixture containing ascorbic acid and 2-keto-L-gulonic acid.

We have found that this object is achieved by the embodiments described herein and characterized in the claims.

The present invention accordingly relates to a process for extracting 2-keto-L-gulonic acid (KGA) from a polar solvent, which process comprises the following step:
(a) extraction of the 2-keto-L-gulonic acid from the polar, preferably aqueous, solvent with an extractant 1 comprising a tertiary amine of the formula 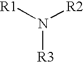
   where R1, R2 and/or R3 is in each case a saturated unbranched or branched alkyl radical having, independently of one another or simultaneously, 6 to 14 carbon atoms;
   and a polar organic diluent;
   and where the extractant 1 has a miscibility gap with the solvent.

In DE 38 31 071, KGA is extracted in the presence of from two to six mole equivalents of a long-chain amine with a CO₂ partial pressure of from 10 to 60 bar.

In EP 359 645, a dilute solution of KGA is extracted with an equal volume of a solution of an amine (Adogen 83) in kerosene, and is back-extracted with nitric acid.

GB 1,426,018 describes the isolation of, inter alia, citric acid, lactic acid and oxalic acid from aqueous solutions by means of extraction.

Building on these, EP 828 725 discloses a process for the extraction of ascorbic acid from an aqueous solution with addition of an acid, using a water-immiscible composition which comprises (a) at least one secondary or tertiary alkylamine in which the total number of carbon atoms is at least 20, as primary extractant and (b) a polar extraction enhancer compound (“enhancer”). The ratio of amine to enhancer in this case is at least 1:2.

Surprisingly, it is now possible through making available the process of the invention to extract KGA selectively from a polar solvent. It is advantageous that it is possible via the process of the invention described herein in particular to separate KGA selectively and economically from ascorbic acid from a polar solvent which contains both KGA and ascorbic acid in solution. It has not to date been shown that the two similar organic acids ascorbic acid and KGA can be selectively separated from one another by a liquid/liquid extraction.

Accordingly, in a particularly preferred embodiment, KGA is advantageously extracted from a polar solvent which comprises ascorbic acid and KGA.

Economic separation by extraction is possible when there is a miscibility gap between the extractant and the solvent, and the difference between the partition coefficients for the substances to be separated, in this case of ascorbic acid and KGA, in one extractant is sufficiently large. Because of the structural similarity of ascorbic acid and KGA, it was not to be expected that it would be possible to find an extractant for which a sufficiently different but also sufficiently high partition coefficient in relation to a polar solvent, especially water, exists. This is also shown by the fact that, although the advantages of a partial autocatalytic lactonization and dispensing with catalysts have been known since 1940, this process step is not used on the industrial scale for preparing ascorbic acid because of the lack of suitable processes for separating precursor and product.

The term “extraction” or “extracting” hereinafter means according to the invention that the substances present in a solid or liquid sample with nonpolar to polar solvents or solvent mixtures, especially ascorbic acid or KGA, are transferred therefrom into the particular extractant or extractant mixture. Extractant also means hereinafter a mixture of various solvents as long as the mixture has the properties described herein for the extractant, in particular can serve as extractant for ascorbic acid or KGA.

The extraction according to the invention is a “liquid/liquid extraction”. A “liquid/liquid extraction” means according to the invention an extraction of a substance dissolved in a liquid solvent by means of a second liquid solvent. The extraction compositions, e.g. the extractant or the temperature, can be chosen so that a specific substance is essentially or preferably extracted or not extracted.

Polar solvents are according to the invention aqueous solutions, including water, or polar aprotic or protic organic solvents, for example alkyl alcohols with an alkyl radical having 1 to 4 carbon atoms, e.g. methanol, ethanol, 1-propanol, 2-propanol or butanol or, for example, acetone, acetonitrile or dimethyl sulfoxide, or they are mixtures thereof.

The term “aqueous solution” means water or an aqueous solution, including, for example, deionized, demineralized, distilled or double-distilled water. One or more substances may be dissolved in aqueous solution or mixed therewith. Thus, substances which improve the extraction, stability or solubility of the substances of value, or lead to preferred properties, e.g. pH, conductivity, salt concentration etc., may be present, such as, for example, salt or buffer solutions.

The solvent preferably comprises a KGA content as described hereinafter. In a preferred embodiment in which the solvent also comprises ascorbic acid, the KGA content and the ascorbic acid content is as described hereinafter.

“Extractant 1” means according to the invention a solvent or solvent mixture which is not miscible with the solvent and has a miscibility gap with the solvent. Thus, extraction results in a phase 1 which comprises KGA-loaded extractant, and a phase 2 which comprises the solvent and, where appropriate, ascorbic acid.

Extractant 1 preferably essentially comprises the tertiary amine of the invention of the formula 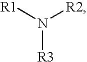
where R1, R2, and/or R3 is in each case a saturated unbranched or branched alkyl radical having, independently of one another or simultaneously, 6 to 14 carbon atoms, and R1, R2, and/or R3 can in particular be —(CH₂)ₙ—CH₃ with n in each case equal to 6 to 14, or consists of a mixture of the amines of the invention, and the organic polar diluent. The extractant preferably consists only of said amines or a mixture thereof and the organic polar diluent.

An alkyl radical having 8 to 12 carbon atoms is particularly preferred, and n is thus in particular preferably equal to 8 to 12. In a particularly preferred embodiment, R1 equals R2 equals R3. Accordingly, the process of the invention relates in a particularly preferred embodiment to extraction with an extractant which comprises tri-n-octylamine and/or tri-n-decylamine.

The term “diluent” means according to the invention also the polar, in particular protic, enhancers disclosed in EP 828 725, especially alkanols, ketones, aldehydes, esters and ethers. The polar organic diluent present in the extractant preferably consists of a saturated branched or unbranched alkyl alcohol having 4 to 14 carbon atoms. The diluent is preferably a saturated branched or unbranched alkyl alcohol having 8 to 12 carbon atoms, and it is very preferably i- or n-decanol, or a mixture thereof.

Extractant 1 thus preferably consists of tri-n-octylamine and tri-n-decylamine, in particular in the ratio from 1:0 to 0:1, preferably in the ratios from 30:60 to 60:30, and of the diluent, especially decanol. Such amine mixtures are commercially available under the proprietary name Hostarex.

The preferred ratio of amine to diluent depends on the particular components. The ratio is preferably from 20:80 to 80:20. It is preferred to use a mixture which comprises tri-n-octylamine/tri-n-decylamine together with a C₈- to C₁₂-alkyl alcohol, preferably n- or i-decanol, preferably in a ratio of tri-n-octylamine/tri-n-decylamine to n- or isodecanol of from 20:80 to 80:20. A particularly preferred ratio of tri-n-otylamine/tri-n-decylamine to isodecanol is 40/60.

Extraction with the following components and proportions is most preferred: amine: tri-n-octylamine/trin-decylamine 50:50 and amine to isodecanol: 40:60.

Separation of KGA from a mixture of ascorbic acid and KGA is possible economically according to the invention when the ratio of the partition coefficients for KGA to ascorbic acid under normal conditions is at least 1.5:1, preferably 4:1, more preferably 7:1 or more, the partition coefficient naturally being dependent on the temperature. The partition coefficient can be determined by methods familiar to the skilled worker, e.g. by a one-stage extraction with subsequent HPLC analysis and iodometric titration.

The extraction of the invention can be carried out as described in the documents cited herein or as described in the examples, e.g. by means of a countercurrent extraction column or of a multistage mixer/decanter cascade (mixer-settler).

Is is preferred in the process of the invention for extractant 1 and the mixture of ascorbic acid and KGA in the solvent to be employed in a ratio of from 0.5:1 to 3:1, with a ratio of from 2:1 to 1:1 being preferred, and a ratio of 1:1 being particularly preferred.

In one embodiment, an aqueous solution or a branched or unbranched C₁- to C₄-alkyl alcohol is used as solvent in the process of the invention. Water or an aqueous solution is preferably used. The term “aqueous solution” encompasses according to the definition used herein both water and buffers, fermentation solutions, salt solutions and other solutions which comprise substances in order to influence for example the pH, the sterility of the solution or the stability of the substances. The solvent may also be a fermentation broth or a supernatant of the decanted, filtered or otherwise purified fermentation broth.

Before the extraction of the KGA in step (a) it is possible in one embodiment to concentrate the product discharge from the previous lactonization reaction, as described below for example. Thus, the concentration is advantageously followed by cooling of the solution and then extraction of the KGA. The concentration advantageously takes place by evaporation at elevated temperature and under reduced pressure, e.g. as described herein.

In a very particularly preferred embodiment, water or an aqueous solution, e.g. a fermentation broth, e.g. with the proportions of KGA and ascorbic acid described below, is used as solvent, and tri-n-otylamine/tri-n-decylamine/i-decanol in the ratio 20:20:60 is used as extractant 1, in the process of the invention.

The extraction in step (a) of the process of the invention preferably takes place at a temperature between 10° C. and 60° C. A temperature between 15° C. and 30° C. is particularly preferred. The skilled person will in choosing the preferred temperature take into consideration the extraction efficiency versus the cooling energy input to achieve the particular extraction temperatures, and the solubility of the precursors at the particular extraction temperatures. Economic and ecological reasons may mean that a preferred temperature is one which can be reached without additional energy input for cooling or heating (ambient temperature). The process step of the invention is most preferably carried out at from 30° C. to 60° C., preferably at 40° C.

In a further embodiment, the method of the invention comprises the following further step:
(b) complete or partial back-extraction of ascorbic acid or KGA from the loaded extractant 1 with a polar extractant 2, resulting in a KGA-loaded extractant 2.

“Complete or partial back-extraction” means according to the invention that KGA is substantially, preferably at least to 30% by weight to 100% by weight, back-extracted into extractant 2. 50% by weight is preferred, and 75% by weight or more is more preferred.

In order to make efficient back-extraction possible, the KGA concentration in extractant 2 is lower than in extractant 1 before the back-extraction, i.e. the proportion is preferably 10% by weight, more preferably 5% by weight or 1% by weight or less, most preferably 0.1 or fewer % by weight.

Extractant 2 is a polar solvent as described above, and is preferably water or an aqueous solution or a branched or unbranched C₁- to C₄-alkyl alcohol.

In a preferred embodiment, extractant 2 and the solvent consist substantially of the same solvent components.

“Consisting substantially of the same solvent components” means in this case that the two compositions do not differ substantially in their solvent constituents, e.g. are 30% or less, more preferably are 10%, even more preferably are 5% different or an identical composition. Thus, for example, one composition may consist substantially of an aqueous solution with a small proportion of an alkyl alcohol, while the other composition consists only of an aqueous solution. In a preferred embodiment, the two compositions are identical in terms of their solvent components and proportions.

Extractant 2 is preferably likewise polar. Solvent and extractant 2 are particularly preferably water or aqueous solutions.

In one embodiment, solvent and extractant 2 consist of solutions comprising substantially the same substances apart from the proportion of ascorbic acid and KGA.

“Comprising substantially the same substances apart from the proportion of ascorbic acid and KGA” means that the two compositions are substantially identical in the proportion of dissolved and undissolved constituents apart from ascorbic acid and KGA and differ only slightly, and preferably 30%, even more preferably 5% or less, of the constituents apart from KGA or ascorbic acid are different.

In a preferred embodiment, the extraction temperature T₁ in the process of the invention for the extraction of the KGA from the solvent which comprises a mixture of ascorbic acid and KGA is 5° C. to 100° C. lower than the back-extraction temperature T₂ for the back-extraction of the KGA from extractant 1 with extractant 2. The difference is preferably from 15° C. to 70° C., and more preferably from 20° C. to 40° C.

As shown in GB 1,426,018, it is possible to reach a higher concentration in the back extract on back-extractions with higher temperatures than in the first extraction using the same solvent, such as, for example, on extraction at room temperature and back-extraction at 100° C.

Consequently, in one embodiment of the present invention, the temperature of extraction is 10 ° C. to 30° C. and the back-extraction temperature is 20° C. to 80° C. It is preferred to combine ambient temperature or room temperature, this meaning a temperature of from 15° C. to 30° C., for the extraction with a temperature of from 40° C. to 60° C. for the back-extraction.

In one embodiment, the process of the invention also comprises the following step:
(c) recycling of extractant 1 from which the KGA was back-extracted in step (b) into the extraction of step (a).

It is preferred for the extractant before recycling and reuse as extractant in step (a) to be partially or completely discharged, worked up and only then returned. Impurities are removed by the discharging. The extractant can be purified for example by distillation, microfiltration or nanofiltration or adsorption (e.g. on activated carbon).

The proportion of discharged material depends substantially on the purity of solvent 1 and the proportion of back-extracted product of value, i.e. of KGA in extractant 1 after back-extraction has taken place. If, after the back-extraction with extractant 2, the extractant comprises only small proportions of product of value and a high proportion of impurities, it is possible to discharge a large proportion of contaminated extractant 1. If there is only partial back-extraction in the back-extraction, a high proportion of product of value is still present in extractant 1, and the skilled worker will routinely consider the loss due to discharge versus the degree of contamination.

The process of the invention for extracting KGA preferably comprises the following further step:
(d) recycling of the KGA-loaded extractant 2 from the back-extraction in step (b) into a process for preparing ascorbic acid from KGA.

The KGA-loaded solvent is advantageously returned to a lactonization step where it is converted into ascorbic acid. The lactonization product discharge can then be subjected to the steps, described herein, of the process of the invention or to the steps of another process known to the skilled worker for isolation of the ascorbic acid.

In one embodiment, the process of the invention comprises the following further step:
(e) concentration of the KGA-loaded extractant 2 before the recycling in step (d); and optionally the step
(f) recycling of the vapors from an evaporation in (e) as extractant 2 in step (b).

“Concentration” means herein that the volume of the sample is reduced and the concentration of KGA or ascorbic acid after the concentration is higher than in the initial solution without, however, precipitating. Thus, 10% or more of the volume of the solvent can be removed. The loaded extractant 2 is preferably stripped or evaporated as far as the solubility limit of ascorbic acid. In a preferred embodiment, the amount of solvent evaporated is exactly enough to allow stationary states to be set up in the continuous system with recyclings. Consequently, “evaporation” means herein a “concentration”.

A concentration can take place for example by heating, especially under reduced pressure, for example circulating evaporator, thin film evaporator etc. Samples can likewise be concentrated by dialysis. The concentration should take place under mild conditions, preferably at from −20° C. to 100° C., depending on the reaction time, pressure and solvent. Concentration at 30° C. to 90° C. is preferred, and at 30° C. to 50° C. is particularly preferred. It is advantageous for the concentration to be carried out under reduced pressure. Depending on the solvent or solvent mixture, the concentration can be carried out under atmospheric pressure (1013 mbar) down to 10 mbar. In the case of aqueous solutions, concentration is preferably at 500 mbar to 10 mbar. The solvent can be cooled after the evaporation, e.g. to ambient temperature or temperature of the following process step, e.g. by means of heat exchangers.

The concentration in the process of the invention advantageously takes place by evaporation of the solvent at 30° C. to 50° C. under a pressure of from 50 to 80 mbar. After the concentration, the solution can be cooled where appropriate and only then fed to the lactonization reactor.

The vapors from the concentration consists substantially of extractant 2 and can therefore advantageously be used again as extractant 2 in step (b).

If, in the process of the invention for separating ascorbic acid and KGA in a mixture, KGA is transferred by extraction into the extractant, the substantial proportion of ascorbic acid remains in the solvent. To isolate the ascorbic acid it is possible in a preferred embodiment of the present invention for the process to comprise the following further step:
(j) isolation of the ascorbic acid from the ascorbic acid-loaded solvent, with a mother liquor remaining behind, preferably by crystallization.

The skilled worker is aware of various process steps for isolating ascorbic acid from polar solvents. Thus, for example, evaporation, cooling or displacement crystallizations or various drying processes, e.g. spray drying for carboxylic acids, especially also for ascorbic acid, are described. For isolation of ascorbic acid it is likewise possible to form insoluble salts or derivatives which then precipitate in the solvent. Ascorbic acid is preferably isolated by an evaporation, cooling or displacement crystallization.

It may therefore be advantageous first to concentrate the ascorbic acid-loaded solvent. Consequently, the process of the invention may in one embodiment comprise at least one of the following further steps before step (j):
(g) washing of the KGA-loaded extractant 1 with the solvent or with the mother liquor from the crystallization of ascorbic acid from the solvent and combining of the ascorbic acid-containing wash solution with the ascorbic acid-loaded solvent in step (a); and
(h) concentration of the ascorbic acid-loaded solvent 1 after extraction of the KGA in step (a).

The washing can take place in the upper part of the extraction column in which the extraction can be carried out.

The concentration takes place as has been described above. The concentration particularly advantageously takes place by evaporation of the solvent at 30° C. to 50° C. and under reduced pressure, with a temperature of 40° C. and a pressure of from 50 to 100 mbar being more advantageous.

Since the mother liquor left behind in step (j) on crystallization of the ascorbic acid may still comprise ascorbic acid, the process of the invention comprises in a preferred embodiment also one of the following further steps:
(i) recycling of the solvent discharge from step (h) into the back-extraction in step (b) as extractant 2.
(k) recycling of the mother liquor into the concentration after step (h).

Solvent is obtained in the solvent discharge from the ascorbic acid evaporation and can then be used as extractant 2 for back-extraction of the KGA from the extractant in step (b). The solvent consumption in the process is reduced thereby.

It would be possible with the process described herein to remove KGA from a mixture also composed of ascorbic acid, KGA, monoacetone-2-keto-L-gulonic acid and/or diacetone-ketogulonic acid.

The present invention also relates in one embodiment to a process for preparing ascorbic acid from 2-keto-L-gulonic acid, which comprises the following steps:
i. lactonization, preferably partial lactonization, of 2-keto-L-gulonic acid;
ii. extraction of the KGA from the ascorbic acid/KGA mixture by the process described herein; and
iii. isolation of the ascorbic acid from the ascorbic acid-loaded solvent.

The mixture of KGA and ascorbic acid can be prepared by processes known to the skilled worker, e.g. by a process described herein for the lactonization of KGA. The mixture is preferably prepared by a direct partial lactonization, in particular by an autocatalytic lactonization of KGA to ascorbic acid.

“Partial lactonization” means according to the invention an incomplete conversion of the precursor into ascorbic acid. The conversion of the precursor into ascorbic acid in the process of the invention is preferably from 10% by weight to 95% by weight, more preferably 20% by weight to 50% by weight. An embodiment with a partial KGA conversion of from 20% by weight to 40% by weight is particularly preferred.

The lactonization reaction (i) can be carried out via processes as described in the prior art since 1933, as long as a mixture of the precursor, preferably KGA, and ascorbic acid in a polar solvent, preferably in an aqueous solution, especially water, is obtained. Owing to the lack of separation processes, the literature ordinarily describes complete conversions of KGA to ascorbic acid or, in the case of only partial conversion, links the separation with the derivatization of the KGA to an ester and following crystallization of the ascorbic acid as described above.

Processes for lactonization are described in the abovementioned prior art and the documents cited therein, which are hereby expressly included in the disclosure content of this description.

It is now possible for the first time by the process described herein to convert KGA only partially into ascorbic acid in a lactonization reaction, for example under mild conditions through short lactonization times or autocatalytically, and then to separate ascorbic acid and KGA selectively.

It would also be possible to use the process described herein to separate ascorbic acid from other starting materials. Ascorbic acid is normally prepared from 2-keto-L-gulonic acid, monoacetone-2-keto-L-gulonic acid or diacetone-ketogulonic acid. Other precursors such as, for example, L-gulono-γ-lactone and the sodium salt of the α-alkyl-KGA pyranoside have also been described.

Direct lactonizations are usually catalyzed by acid, preferably with hydrochloric acid as gas or with aqueous hydrochloric acid, and have long been known in the state of the art.

In processes catalyzed by alkali, the lactonization reaction rate is higher, leading to higher space-time yields in the apparatuses. Basic catalysts used are, besides NaOH in various alcohol or alcohol/water mixtures, alkali metal salts of weak acids (e.g. NaHCO₃ or sodium acetate), Na₂CO₃ or sodium methoxide in alcohols. The initial product of these processes is the sodium salt of ascorbic acid, which must be converted into free ascorbic acid in a further process step. A process for preparing free ascorbic acid is described in U.S. Pat. No. 5,041,563.

It is necessary in the acidic processes mentioned to remove the catalyst. The acid may decompose the product. With alkaline catalysis there is initial preparation of an ascorbic acid salt, which must be converted into free ascorbic acid.

Catalyst-free lactonization of KGA and KGA esters to ascorbic acid by simple heating in water, alcohols or mixtures of water with a hydrophilic solvent at temperatures above 130° C. and with residence times of from 30 minutes to 90 hours has also been described since about 1940. Addition of citric acid and phosphate as buffer to set a constant pH is said to be able to increase the yields.

It is now possible in an advantageous manner by the process of the invention to carry out a direct acid- or alkali-catalyzed or autocatalytic partial lactonization, e.g. on an acidic ion exchanger (e.g. Bayer Lewatit) or, preferably, by means of fixed bed catalysis. The lactonization is preferably carried out at low temperatures which lead to little derivatization or decomposition of the resulting ascorbic acid, particularly preferably below 60° C., e.g. by means of biocatalysis or enzymic catalysis or in acidic catalysis.

Thus, in a particularly preferred embodiment, step (i) for lactonization of 2-keto-L-gulonic acid takes place autocatalytically and partially in the process of the invention.

The lactonizations in most processes are carried out with complete conversion of the particular precursor. The advantage of autocatalytic conversion is that neither catalysts nor other auxiliaries are required and need to be removed from the reaction discharge. Economic use of autocatalytic lactonization has failed to date because complete conversion takes place inefficiently and with low yields. A suitable separation process for isolating ascorbic acid from a mixture of KGA and ascorbic acid, as is obtained by a partial conversion, had not been described and is made available for the first time in the present invention.

It is known that KGA can be lactonized in aqueous solutions by exposure to elevated temperature (T>25° C., T<200° C.). Temperatures of from 40 to 180° C. are preferred. It is advantageously possible in such a way to achieve a very short conversion time in the reactor. If a solution of KGA in water is heated at 80-150° C., and the residence time in the reactor is kept between 1 and 30 min, it is possible with KGA conversions of 25-30% to obtain ascorbic acid selectivities of around 90% in solution. Partial conversion with precursor recycling has previously been described only in the case of the KGA esters. The initial concentration of KGA in water preferably does not exceed 30%.

Consequently, the present invention also relates to a process for preparing and isolating ascorbic acid, where step (i) for lactonization of 2-keto-L-gulonic acid is carried out autocatalytically with partial conversion under the following conditions:
(aa) at a temperature of from 60° C. to 180° C., preferably between 100° C. and 160° C.;
(bb) with an initial mass fraction of 2-keto-L-gulonic acid of from 5% by weight to 50% by weight, preferably between 10% and 15%;
(cc) with a KGA conversion of from 10 to 40% by weight, preferably 20 to 30% by weight; and/or
(dd) with a residence time in the lactonization reactor of from 1 to 30 min, preferably 10 min or less.

Particular preference is given to an initial mass fraction of KGA of from 10 to 15%, a reactor temperature of from 110° C. to 150° C. with a residence time of from 3 to 5 min and a KGA conversion of from 20 to 25% by weight.

Examples of reactors suitable for the lactonization are tube bundles, plate heat exchangers, helical tube reactor or jet reactors.

The reaction discharge from the lactonization reaction is concentrated to achieve a stationary state of operation in accordance with the concentration steps described above. It is then possible to remove the ascorbic acid or the KGA as in step (a) from the reaction discharge which has preferably been cooled to ambient temperature or 20° C. to 25° C.

The reaction discharge preferably has a KGA content after the concentration of from 5 to 30% by weight, particularly preferably 8 to 25% by weight, and an ascorbic acid content of from 3 to 20% by weight, particularly preferably 5 to 10% by weight. Consequently, the KGA-containing solvent in step (a) also has a KGA content of from 5 to 30% by weight, particularly preferably 8 to 25% by weight. In a particularly preferred embodiment, the solvent in step (a) has a KGA content of from 5 to 30% by weight, particularly preferably 8 to 25% by weight, and an ascorbic acid content of from 3 to 20% by weight, particularly preferably 5 to 10% by weight.

To isolate the ascorbic acid, the ascorbic acid-loaded solvent is preferably concentrated and the ascorbic acid is crystallized from the solvent.

In a preferred embodiment, the condensed vapors from the various evaporation steps in the process of the invention substantially remain in the process and are employed therein as solvent, as has been described above for the various process steps. It is particularly preferred for the evaporation of the respective solvents to be operated via the respective operating pressure so that energy can be transferred from the vapor condenser of a first evaporation to the evaporator of a second evaporation.

It is possible according to the invention for the individual steps of the process described herein to be carried out continuously or batchwise. The preferred embodiment is for the steps to be carried out continuously.

In one embodiment, the process of the invention for the isolation of ascorbic acid or for the preparation of ascorbic acid comprises all the steps (a) to (g) and/or (i) to (iii) and/or (aa) to (cc) described herein. This advantageously results in ascorbic acid and/or KGA without production of salts.

The present invention is illustrated by the following example without this being intended in any way to be regarded as restrictive.

### EXAMPLES

In a laboratory experiment, an aqueous solution comprising 5% by weight ascorbic acid and 10% by weight KGA was extracted with a 40/60 Hostarex/i-decanol mixture at 30° C. and with a ratio of extractant 1 to solvent in which KGA and ascorbic acid was present in said concentration of 1 kg/kg.

The partition coefficient (ratio of the concentration of recipient/donor phase) measured for ascorbic acid was 1.1 kg/kg and for KGA was 6.6 kg/kg. Accordingly, the ratio of the partition coefficients, i.e. the selectivity, equals 6.

Concerning the back-extraction of KGA from extractant 1, it was found that the partition coefficient of KGA is reduced to 0.18 at 80° C. under otherwise comparable conditions.

This means that a comparatively high partition coefficient of 5.5 (reciprocal of 0.18) is possible for the back-extraction of KGA with extractant 2 (water) from extractant 1. The specific management of temperature thus makes economic extraction/back-extraction of KGA possible.

1. A process for extracting 2-keto-L-gulonic acid (KGA) from a polar solvent comprising ascorbic acid and 2-keto-L-gulonic acid, which process comprises the following step:
(a) extraction of the 2-keto-L-gulonic acid from the polar solvent with an extractant 1 comprising a tertiary amine of the formula 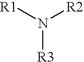
where R1, R2 and/or R3 is in each case a saturated unbranched or branched alkyl radical having, independently of one another or simultaneously, 6 to 14 carbon atoms;
and a polar organic diluent; where the diluent is a saturated branched or unbranched alkyl alcohol having 8 to 12 carbon atoms or an amide or an aromatic compound,
and where the extractant 1 has a miscibility gap with the solvent.

2. A process as claimed in claim 1, where the alkyl radical R1, R2 and/or R3 comprises in each case 8 to 12 carbon atoms.

3. A process as claimed in claim 1, where the tertiary amine is tri-n-octylamine and/or tri-n-decylamine.

4. A process as claimed in claim 1, where the diluent is i- or n-decanol.

5. A process as claimed in claim 1, where the ratio of tertiary amine of the formula I to the diluent is from 20:80 to 80:20.

6. A process as claimed in claim 1, comprising the following further step:
(b) back-extraction of the KGA from the loaded extractant 1 with a polar extractant 2, resulting in a KGA-loaded extractant 2.

7. A process as claimed in claim 6, where extractant 2 and the solvent consist essentially of the same solvent components.

8. A process as claimed in claim 7, where the extraction temperature T₁, is from 5° C. to 100° C. lower than the back-extraction temperature T₂.

9. A process as claimed in claim 6, comprising the following further step:
(c) recycling of extractant 1 from which the KGA was back-extracted in step (b) into the extraction of step (a).

10. A process as claimed in claim 6, comprising the following further step:
(d) recycling of the KGA-loaded extractant 2 from the back-extraction in step (b) into a process for preparing ascorbic acid from KGA.

11. A process as claimed in claim 10, comprising the following further steps:
(e) concentration of the KGA-loaded extractant 2 before the recycling in step (d); and
(f) optionally, recycling of the vapors from the evaporation in (e) as extractant 2 in step (b).

12. A process as claimed in claim 11, comprising at least one of the following further steps:
(g) washing of the KGA-loaded extractant 1 with the solvent or with the mother liquor from the crystallization of ascorbic acid from the solvent and combining of the ascorbic acid-containing wash solution with the ascorbic acid-loaded solvent in step (a);
(h) concentration of the ascorbic acid-loaded solvent 1; and
(i) recycling of the solvent discharge from step (h) into the back-extraction in step (b) as extractant 2.

13. A process as claimed in claim 12, comprising the following further steps:
(j) isolation of the ascorbic acid from the ascorbic acid-loaded solvent, with a mother liquor remaining behind; and
(k) optionally, recycling of the mother liquor from step (j) into the concentration in step (h).

14. A process for preparing ascorbic acid, which comprises the following steps:
i. lactonization of 2-keto-L-gulonic acid;
ii. extraction of the KGA from the ascorbic acid/KGA mixture as set forth in claim 2; and
iii. isolation of the ascorbic acid from the ascorbic acid-loaded solvent.

15. A process as claimed in claim 14, where partial lactonization is carried out.

16. A process as claimed in claim 1, wherein said solvent is aqueous.

17. A process as claimed in claim 2, wherein
the tertiary amine is tri-n-octylamine or tri-n-decylamine or mixtures thereof,
the diluent is i- or n-decanol and
the ratio of tertiary amine of the formula I to the diluent is from 40:60.
